(19) United States
(12) Patent Application Publication
(10) Pub. No.: US 2016/0242521 A1
(43) Pub. Date: Aug. 25, 2016

(54) Hair Retained Device Fastener

(71) Applicants: Luz C. Perez, Teaneck, NJ (US); Ramon D. Perez, Teaneck, NJ (US)

(72) Inventors: Luz C. Perez, Teaneck, NJ (US); Ramon D. Perez, Teaneck, NJ (US)

(21) Appl. No.: 15/051,664

(22) Filed: Feb. 24, 2016

Related U.S. Application Data
(60) Provisional application No. 62/119,791, filed on Feb. 23, 2015.

(51) Int. Cl. A45D 8/34; A61N 1/36

(52) U.S. Cl. CPC A45D 8/34; A61N 1/36032; A45D 2008/004

(57) ABSTRACT

It is an object of the present embodiment of the invention to fasten a device to the hair, in this case the device includes but is not limited to a sound processor for a cochlear implant.

### BACKGROUND

The field of the current embodiment of the invention relates to cochlear implant recipients with respect to securing the processor in place. Especially when it relates to younger children and infants as the implantation surgery can be performed as early as 9 months of age. In this early age group it becomes difficult to maintain the processor of the cochlear implant in place behind the ear due to the fact that they lack the motor skills and awareness to maintain it in place on their own. It is of up most importance to use the processor for the most amount of time possible per day in order for the individual to receive optimal benefit from the implant. The current manners available to try to secure a processor in place include headbands, wig tape and hooks & loops that wrap around the ear.

### SUMMARY

It is hence an object of the present invention to fasten a device to the hair.

It is another object of the present embodiment to provide a comfortable, decorative, secure, and discrete manner to fasten a device to the hair, in this case the device includes but is not limited to a sound processor for a cochlear implant.

It is yet another object of the present embodiment to improve functionality and increase productivity with children in the said age group with cochlear implants by increasing the time that the processor is worn.

### BRIEF DESCRIPTION OF THE DRAWINGS

FIG. 1 is a front view of the present embodiment with single fastener.

FIG. 2 is a side view of the present embodiment with single fastener.

FIG. 3 is a top view of the present embodiment with single fastener.

FIG. 4 is a perspective view of the present embodiment with single fastener.

FIG. 5 is a view of the present embodiment with single fastener, wherein the device is placed through and secured by the elastic loop (2).

FIG. 6 is a view of the present embodiment with single fastener, wherein the device is fastened and secured to the hair and discretely concealed by the decorative element (3).

FIG. 7 is a side view of the present embodiment with multiple fasteners.

FIG. 8 is a top view of the present embodiment with multiple fasteners.

FIG. 9 is a back view of the present embodiment with multiple fasteners.

FIG. 10 is a view of the present embodiment with multiple fasteners, wherein the devices are placed through and secured by the elastic loops (2).

FIG. 11 is a view of the present embodiment with multiple fasteners, wherein the device is fastened and secured to the hair and discretely concealed by the decorative element (3).

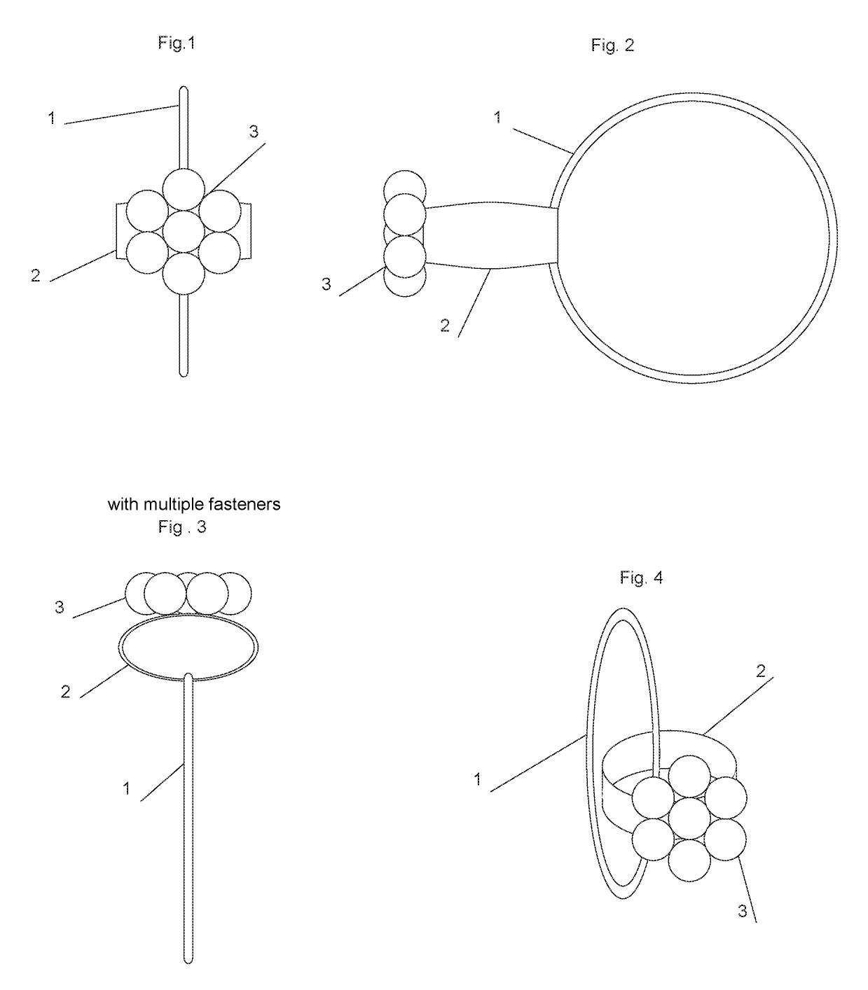

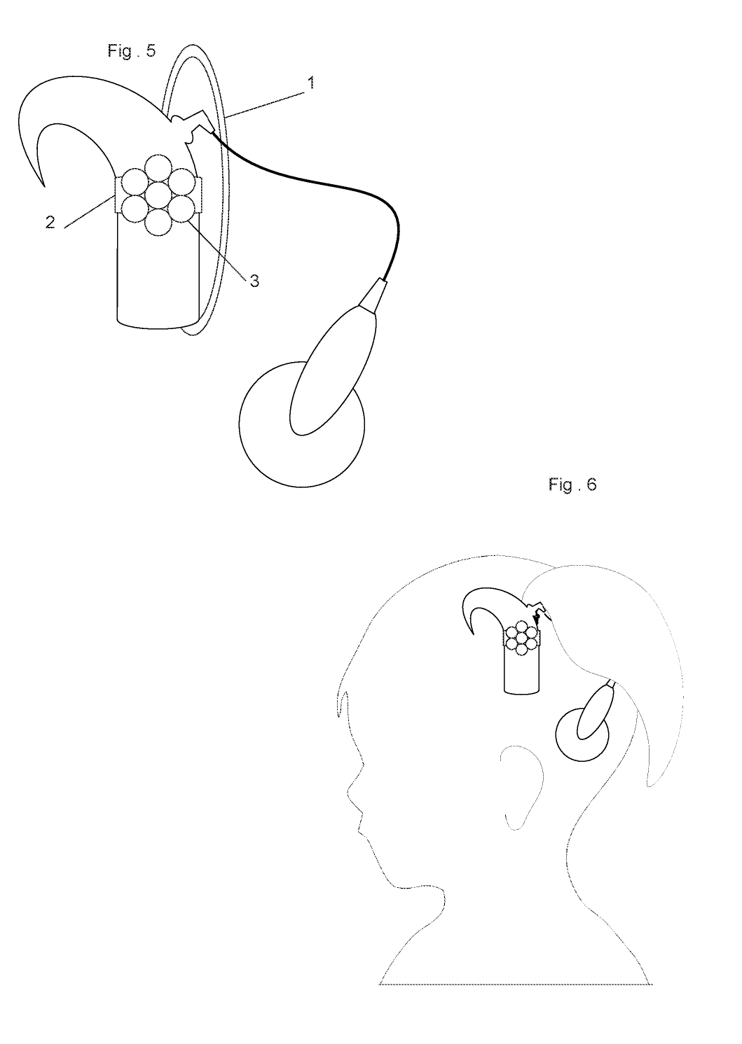

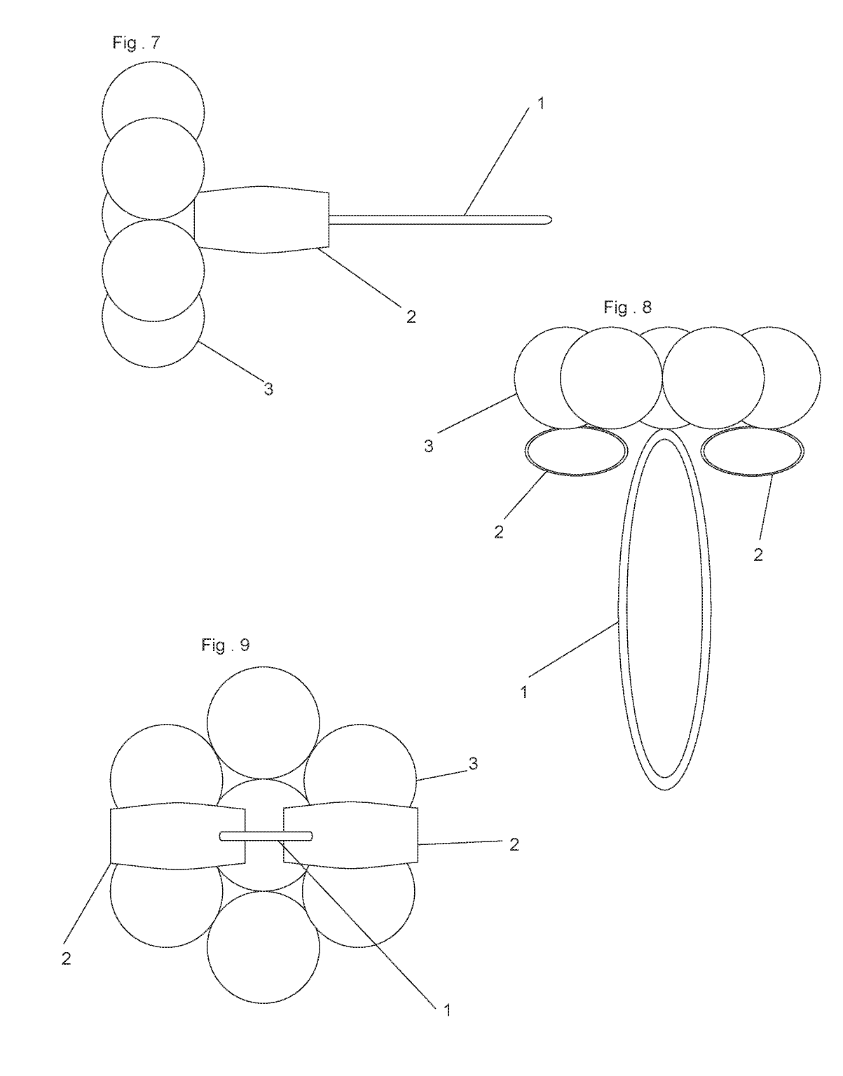

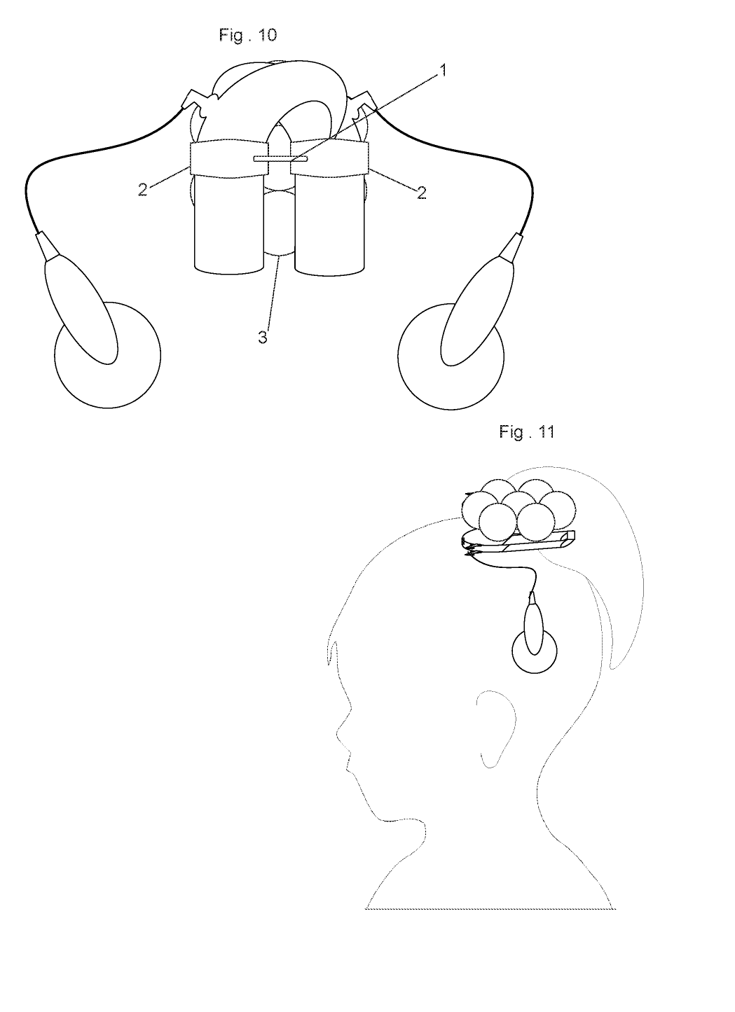

### DETAILED DESCRIPTION

The Hair Retained Device Fastener
A hair fastener which in the present embodiment includes but is not limited to an elastic hair tie (1);
a fastener or multiple fasteners which in the present embodiment include but are not limited to an elastic loop (2) having a first end and second end;
and a decorative element (3) of any type.
In the singe fastener wherein said hair tie (1) is attached to the first end of said elastic loop (2) by means that include but are not limited to stitching. The second end of said elastic loop (2) is attached to the decorative element (3) by means that include but are not limited to stitching. In the multiple fastener wherein said hair tie (1) is attached to the decorative element (3) by means that include but are not limited to stitching. The said elastic loops (2) are attached to the decorative element (3) by means that include but are not limited to stitching. The hair is then pulled through and fastened by said elastic hair tie (1) then the said device is secured and placed through the said elastic loop (2). Where it is now fastened and secured to the hair and discretely concealed by the decorative element (3).

1. An apparatus for fastening devices to the hair, comprising:
(a) a means for attaching to the hair;
(b) a means for fastening a device, attached to the means of attaching to the hair; and
(c) a decorative element, attached to the means of fastening the device.

2. The apparatus in claim 1,
(a) wherein the means for attaching to the hair in the present embodiment includes but is not limited to an elastic tie (1);
(b) wherein the means of fastening the device in the present embodiment includes but is not limited to an elastic loop (2); and
(c) a decorative element (3) wherein the present embodiment incudes but is not limited to a flower.
